(19) United States
(12) Patent Application Publication
(10) Pub. No.: US 2020/0215390 A1
(43) Pub. Date: Jul. 9, 2020

(54) FITNESS MONITORING SYSTEM

(71) Applicant: GREENCOMM CO., LTD., Anyang-si (KR)

(72) Inventor: Ki Chul SHIN, Gunpo-si (KR)

(73) Assignee: GREENCOMM CO., LTD., Anyang-si (KR)

(21) Appl. No.: 16/328,353

(22) PCT Filed: Aug. 30, 2017
(86) PCT No.: PCT/KR2017/009469

§ 371 Date: Feb. 26, 2019

(30) Foreign Application Priority Data
Aug. 31, 2016 (KR) 10-2016-0111907

(51) Int. Cl. A63B 24/00; A61B 5/024; G04G 17/04; G04G 21/02

(52) U.S. Cl. CPC A63B 24/0062; A61B 5/024; A61B 5/681; G04G 21/025; G04G 17/04

(57) ABSTRACT

Disclosed is a fitness monitoring system. A mobile terminal in a system of the present invention connects ends of multiple signals corresponding to a predetermined behavior and stores a determined envelope area. Further, when a signal relating to a user's behavior corresponds to the envelope area, the mobile terminal determines the user's behavior as the predetermined behavior and determines an accuracy or a similarity of the user's behavior by using a degree of matching of the signal relating to the user's behavior to the envelope area.

### FIELD OF THE DISCLOSURE

The present invention relates to a fitness monitoring system.

### DESCRIPTION OF RELATED ART

Modern people are suffering from imbalanced posture, obesity, and other adult diseases due to lack of physical activity and irregular eating habits. Accordingly, interest in health and exercise is increasing, and various exercise equipment or systems have been developed.

Conventionally, users exercise at a gym under the guidance of a health trainer or exercise at home or at a workplace by watching and following an exercise motion replayed on a purchased compact disc (CD) file, digital versatile disc (DVD) file, computer file, or video tape.

In the case of personal training in which a trainer exercises together with a trainee while directly correcting the exercise posture and repetitions of the trainee, the exercise effect is excellent, therefore people who are inexperienced in exercise prefer exercising at the gym. In the case in which a trainee exercises alone without a trainer, the trainee may able to perform continuous movements of a specific exercise in a gym or at home but have difficulty in maintaining a correct posture and fulfilling an assigned number of repetitions, which usually causes the trainee to stop exercising without attaining an expected effect.

Accordingly, having a lesson in a gym under the guidance of a health trainer is preferable in terms of efficiency in exercise but is not easy for busy modern people due to the burden of lesson fees and the hassle of visiting the gym.

Meanwhile, there is a demand for a system in which a health trainer remotely monitors an exercise state of a trainee exercising at home by watching a monitor so that the trainee is prevented from easily giving up exercising due to inaccuracy of a movement and difficulty in exercise. To this end, the system requires a wearable device that determines a sports movement of a trainee, transmits the degree of movement accuracy and the movement repetition number, accumulates exercise data, and recognizes a change in the exercise data.

The conventional healthcare wearable device mostly performs motion recognition using a camera. A system and method for coaching sports movements by capturing and recognizing a motion of a trainee is disclosed in Korean Patent Publication No. 2012-0131342.

However, the disclosure also uses a camera in capturing and recognizing an action of a user, and the accuracy in recognizing an action is reduced depending on the illumination and the exercising speed.

### SUMMARY OF THE INVENTION

The present invention is directed to providing a fitness monitoring system capable of recognizing an action of a user using a signal received from a sensor of a wearable device worn by a user.

In addition, the present invention is directed to providing a fitness monitoring system capable of determining the degree to which an operation of a user deviates from a reference operation.

One aspect of the present invention provides a fitness monitoring system including a wearable device worn on a predetermined portion of a body of a user to transmit a signal related to an action of the user and a mobile terminal configured to determine the action of the user on the basis of the signal related to the action of the user, wherein the mobile terminal includes a storage unit configured to store an envelope area that is determined by connecting ends of a plurality of signals corresponding to a predetermined action and a control unit configured to determine the action of the user as the predetermined action when the signal related to the action of the user corresponds to the envelope area.

The signal related to the action of the user may be an acceleration signal.

The control unit may check and determine whether a plurality of actions each correspond to the envelope area and determine the action of the user on the basis of a combination of the plurality of actions.

The control unit may determine a degree of accuracy or similarity of the action of the user using a degree to which the signal related to the action of the user matches with the envelope area.

Another aspect of the present invention provides a fitness monitoring system including a wearable device worn on a predetermined portion of a body of a user to transmit a signal related to an action of the user and a mobile terminal configured to display an image in which a trainer performs an operation, determine the action of the user on the basis of the signal related to the action of the user, and determine a degree of accuracy or similarity of the action of the user, wherein the mobile terminal includes a display unit; a storage unit configured to store the image in which the trainer performs the operation and an envelope area of a signal corresponding to the operation of the trainer; and a control unit configured to control the display unit to display the image of the trainer and determine a degree to which the signal related to the action of the user received from the wearable device matches with the envelope area.

The signal related to the action of the user may be an acceleration signal.

Another aspect of the present invention provides a fitness monitoring system including a display apparatus; a wearable device worn on a predetermined portion of a body of a user to transmit a signal related to an action of the user; and a mobile terminal configured to transmit an image in which a trainer performs an operation to the display apparatus, determine the action of the user on the basis of the signal related to the action of the user, and determine a degree of accuracy or similarity of the action of the user, wherein the mobile terminal includes a storage unit configured to store the image in which the trainer performs the operation and an envelope area of a signal corresponding to the operation of the trainer; a communication unit configured to transmit the image of the trainer to the display apparatus; and a control unit configured to determine a degree to which the signal related to the action of the user received from the wearable device matches with the envelope area.

The signal related to the action of the user may be an acceleration signal.

Another aspect of the present invention provides a fitness monitoring system including a wearable device worn on a predetermined portion of a body of a user to collect a signal related to an action of the user, determine information related to the action of the user on the basis of the signal related to the action of the user, and transmit the information related to the action of the user and including a mobile terminal configured to receive the information related to the action of the user, wherein the wearable device includes a sensor unit configured to collect the signal related to the action of the user; a storage unit configured to store an envelope area that is determined by connecting ends of a plurality of signals corresponding to a predetermined action; and a control unit configured to determine the action of the user as the predetermined action when the signal related to the action of the user corresponds to the envelope area.

The control unit may check and determine whether a plurality of actions each correspond to the envelope area and determine the action of the user on the basis of a combination of the plurality of actions.

The control unit may determine a degree of accuracy or similarity of the action of the user using a degree to which the signal related to the action of the user matches with the envelope area.

According to the present invention, a predetermined operation can be identified from a signal related to an action of a user depending on whether the signal falls within a signal range corresponding to the predetermined operation, and the degree of accuracy of the operation can be determined by checking a ratio corresponding to deviation of the signal from the signal range corresponding to the predetermined operation.

Thus, according to the present invention, an operation corresponding to an action of a user can be more accurately identified, and the user is guided to more accurately perform the operation so that the exercise efficiency is enhanced.

### BRIEF DESCRIPTION OF THE DRAWINGS

FIG. 1 is a schematic structural view illustrating a fitness monitoring system according to an embodiment of the present invention.

FIG. 2 is a detailed block diagram illustrating a wearable device according to an embodiment of the present invention.

FIG. 3 is a detailed block diagram illustrating a mobile terminal according to an embodiment of the present invention.

FIG. 4 is a diagram illustrating an acceleration signal shown in a predetermined action.

FIG. 5 is a diagram illustrating a plurality of acceleration signals identical to the acceleration signal shown in FIG. 4 superposed on each other.

FIG. 6 is a diagram for describing an example in which a control unit identifies an action by matching the action with a signal related to an action of a user.

FIG. 7 is a diagram for describing an acceleration pattern corresponding to golf according to an embodiment of the present invention.

FIG. 8 is a diagram for describing an example of implementation according to the present invention.

FIG. 9 is a structural view illustrating a fitness monitoring system according to an embodiment of the present invention.

FIG. 10 is a structural diagram illustrating a fitness monitoring system according to another embodiment of the present invention.

### DETAILED DESCRIPTION OF THE INVENTION

Hereinafter, advantages and features of the present invention and manners of achieving them will become readily apparent with reference to descriptions of the following detailed embodiments when considered in conjunction with the accompanying drawings. However, the scope of the present invention is not limited to such embodiments, and the present invention may be realized in various forms. The embodiments to be described below are provided so that this invention is complete and fully conveys the scope of the present invention to those skilled in the art. The size of each component shown in the drawings may be exaggerated or reduced for the sake of convenience in description.

It will be understood that when an element is referred to as being “on” or “adjacent to” another element, it can be in direct contact or connected with another element or intervening elements may be present. In contrast, when an element is referred to as being “directly on” or “directly adjacent to” to another element, there are no intervening elements present. Other words used to describe the relationship between elements should be interpreted in a like fashion (i.e., “between” versus “directly between”, etc.).

It should be understood that, although the terms “first,” “second,” etc. may be used herein to describe various elements, these elements are not limited by these terms. These terms are only used to distinguish one element from another. For example, a first element could be termed a second element, and, similarly, a second element could also be termed a first element, without departing from the scope of the present invention.

It should be understood that the singular forms “a,” “an,” and “the” are intended to include the plural forms as well, unless the context clearly indicates otherwise. The terms “comprises,” “comprising,” “includes,” and/or “including,” when used herein, specify the presence of stated features, integers, steps, operations, elements, components and/or groups thereof, and do not preclude the presence or addition of one or more other features, integers, steps, operations, elements, components, and/or groups thereof.

Unless otherwise defined, all terms used herein have the same meaning as commonly understood by one of ordinary skill in the art to which this invention belongs.

Hereinafter, preferred embodiments of the present invention will be described with reference to the accompanying drawings.

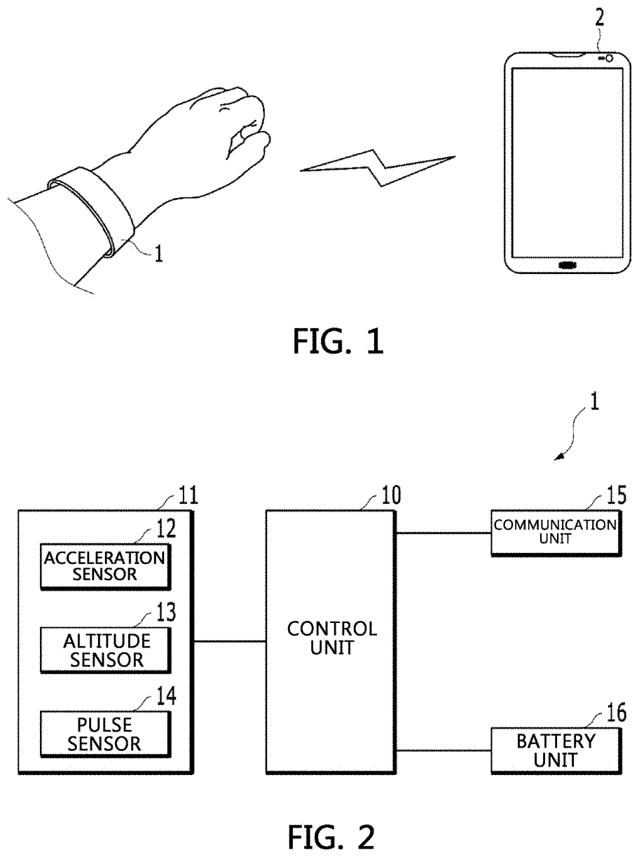

FIG. 1 is a schematic structural view illustrating a fitness monitoring system according to an embodiment of the present invention.

As shown in the drawing, the system according to the embodiment of the present invention includes a wearable device 1 worn on a predetermined portion of a body of a user and a mobile terminal 2.

The wearable device 1 may transmit a signal related to an action performed by the user to the mobile terminal 2. In this case, the wearable device 1 and the mobile terminal 2 may perform communication using short-range wireless communication, such as Bluetooth or near field communication (NFC). This will be described in detail with reference to the drawings.

FIG. 2 is a detailed block diagram illustrating the wearable device according to the embodiment of the present invention.

As shown in the drawing, the wearable device 1 according to the embodiment of the present invention includes a control unit 10, a sensor unit 11, a communication unit 15, a battery unit 16, and a vibrating unit 17. In addition, the sensor unit 11 may include an acceleration sensor 12, an altitude sensor 13, and a pulse sensor 14. However, the components shown in FIG. 2 are not essential, and the number of components forming the wearable device 1 may be less or greater than the number of the components shown in FIG. 2.

The acceleration sensor 12 of the sensor unit 11 may be a three-axis sensor for measuring accelerations of three axes of x, y, and z to distinguish an action of a user and may serve to measure acceleration related to the action of the user.

The altitude sensor 13 is provided to measure an altitude at which the wearable device 1 is currently located and serves to measure atmospheric pressure and convert the measured atmospheric pressure into a voltage.

The pulse sensor 14 may measure a heart rate of the user. Since the heart rate is proportional to the exercise intensity and as a maximum heart rate rises, calorie consumption increases, the exercise intensity may be identified by checking the heart rate during exercise.

In the embodiment of the present invention, the acceleration sensor 12, the altitude sensor 13, and the pulse sensor 14 have been illustrated as an example of a sensor module of the sensor unit 11, but the present invention is not limited thereto and may include other various sensors.

The control unit 10 may transmit a signal related to an action including the acceleration, altitude, and heart rate, which are measured by the sensor unit 11, to the mobile terminal 2 through the communication unit 15. The communication unit 15 may perform communication with the mobile terminal 2 by a short-range communication scheme, such as Bluetooth or NFC, as described above.

The battery unit 16 may supply power to each component of the wearable device 1 according to the present invention and may be implemented as a lithium-polymer battery, for example.

Meanwhile, the mobile terminal 2 is carried by the user or placed adjacent to the user when the user performs an action and serves to receive a signal related to an action performed by the user from the wearable device 1 to recognize the action of the user and determine the degree of accuracy of the action or determine the degree of similarity by which the recognized action is similar to a corresponding operation. In addition, the mobile terminal 2 may provide the user with information about the maximum heart rate, the calorie consumption, and the exercise repetitions. The detailed configuration of the mobile terminal 2 will be described with reference to the drawings.

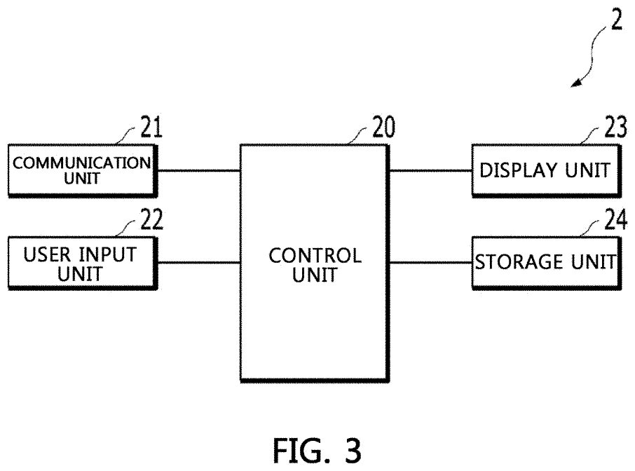

FIG. 3 is a detailed block diagram illustrating a mobile terminal according to an embodiment of the present invention.

As shown in the drawing, the mobile terminal 2 according to the embodiment of the present invention may include a control unit 20, a communication unit 21, a user input unit 22, a display unit 23, and a storage unit 24. However, the components shown in FIG. 3 are not essential, and the number of components forming the mobile terminal 2 may be less or greater than the number of the components shown in FIG. 3.

The communication unit 21 may transmit or receive data to or from the communication unit 15 of the wearable device 1 by a predetermined communication scheme. In this case, the communication unit 21 may perform communication with the wearable device 1 using short-range wireless communication such as Bluetooth or NFC.

The user input unit 22 may generate input data for the user to control an operation of the mobile terminal 2.

The display unit 23 may display information being processed by the mobile terminal 2. The display unit 23 may include at least one of a liquid crystal display (LCD), a thin film transistor-liquid crystal display (TFT-LCD), an organic light-emitting diode (OLED), a flexible display, and a three-dimensional display (3D display).

When the display unit 23 forms a mutual layer structure together with a sensor (a touch sensor) for sensing a touch operation (hereinafter, referred to as a ‘touch screen’), the display unit 23 may be used not only as an output device but also as an input device, i.e., the user input unit 22.

A touch sensor may be configured to convert a pressure applied to a specific portion of the display unit 23 or a change in the electrostatic capacity or the like occurring in a specific portion of the display unit 23 into an electrical input signal. The touch sensor may be configured to detect not only a position and area touched but also a pressure at a time of a touch. In response to a presence of a touch input on the touch sensor, a signal corresponding to the touch input is transmitted to the control unit 20, and the control unit 20 processes the signal so that a touched portion of the display unit 23 is identified.

The storage unit 24 may store a program for the operation of the control unit 20 and an application downloaded by a user for a service according to the embodiment of the present invention. In addition, the storage unit 24 may store a reference parameter such that the control unit 20 compares the reference parameter with an action parameter.

The control unit 20 may receive a signal related to an action of a user through the communication unit 21 and recognize the action of the user and also determine the degree of accuracy or similarity of a specific action of the user. This will be described below with reference to the drawings.

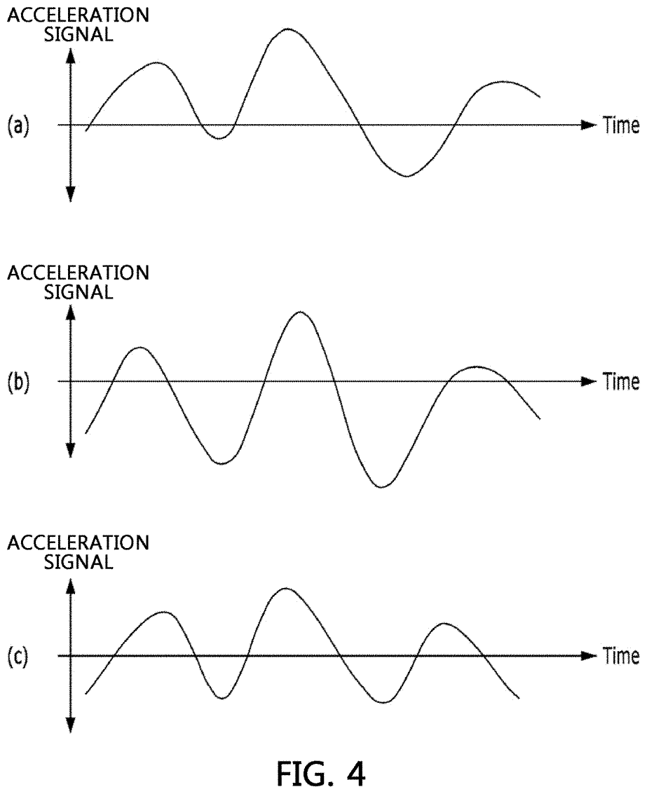

A certain action of a human is accompanied by a series of changes in acceleration. An example of ‘Running’ may be considered. FIG. 4 is a diagram illustrating an acceleration signal shown in a predetermined action. In the embodiment of the present invention, an example of an ‘acceleration signal’ is described, but the present invention is not limited thereto, and the signal may be any type of a signal that can be measured by the sensor unit 11 of the wearable device 1.

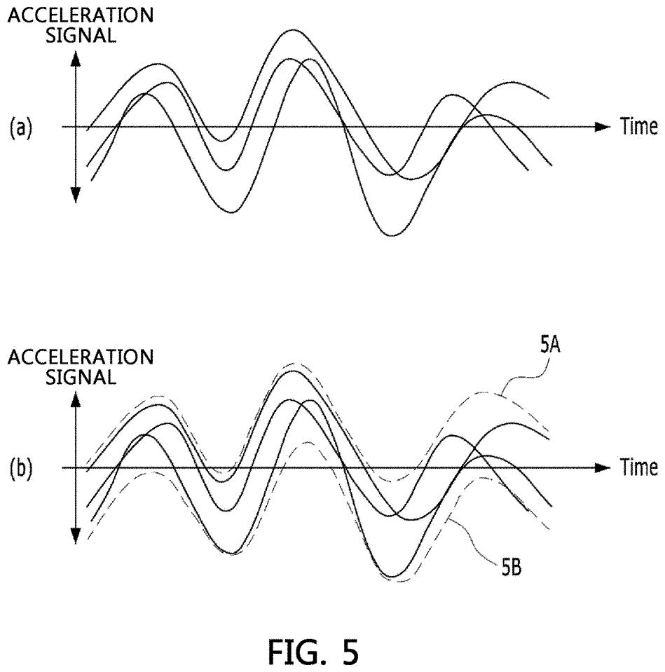

FIGS. 4A to 4C show acceleration signals corresponding to when a plurality of humans wear wearable devices 1 and perform a predetermined action, in which the acceleration signals are provided in various sizes but have similar patterns. FIG. 5 is a diagram illustrating the plurality of acceleration signals identical to the acceleration signal shown in FIG. 4 superposed on each other. Referring to FIG. 5A, it can be seen that the acceleration signals obtained when the plurality of humans wearing the wearable devices 1 perform a predetermined action have similar patterns.

Accordingly, in the embodiment of the present invention, envelopes 5A and 5B may be determined by connecting ends of the plurality of signals forming the superposition signal for the predetermined action as shown in FIG. 5B, and the envelopes may be stored in the storage unit 24. That is, the control unit 20 may receive an envelope of a superposition signal for a predetermined action from a server (not shown) for providing an application according to the embodiment of the present invention and store the received envelope in the storage unit 24. For the sake of convenience in description, hereinafter, the envelope 5A is referred to as an upper envelope and the envelope 5B is referred to a lower envelope. In addition, an area between the upper envelope and the lower envelope is referred to as an ‘envelope area’. As such, the envelope area between the upper envelope and the lower envelope may represent an area for determining the predetermined action on the basis of a signal related to an action of a user.

Such envelopes may be defined for a plurality of actions and may be stored in the storage unit 24. The control unit 20 may receive data related to an action from a server (not shown) whenever the data is updated and store the received data.

That is, the control unit 20 may receive a signal related to an action of a user from the wearable device 1, match the received signal with each of the plurality of envelopes, and identify the action in response to the degree of match greater than or equal to a predetermined ratio.

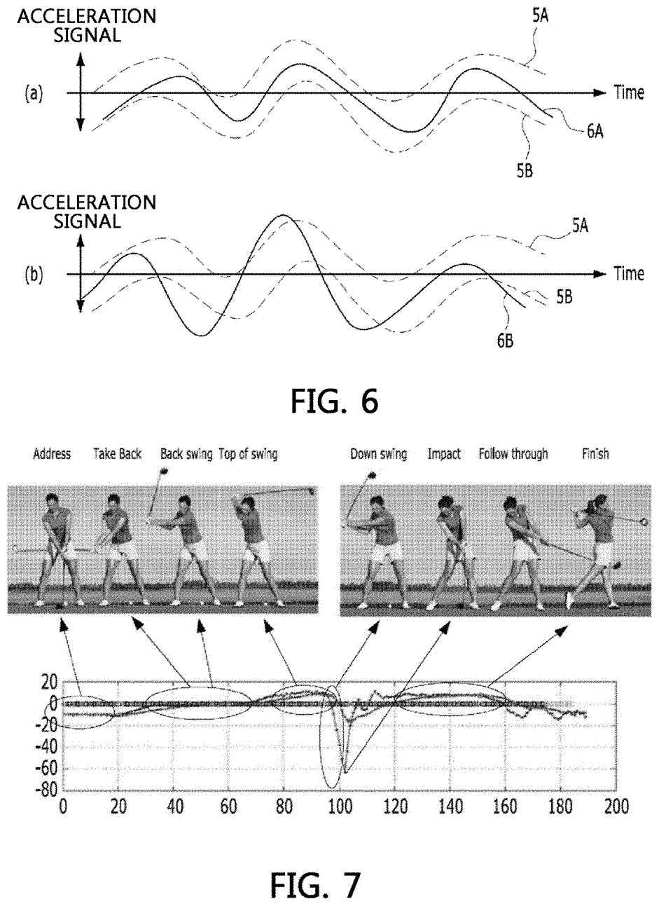

FIG. 6 is a diagram for describing an example in which the control unit identifies an action by matching the action with a signal related to an action of a user. In the embodiment of the present invention, an ‘acceleration signal’ is described as an example of a signal related to an action of a user, but the present invention is not limited thereto.

As shown in FIG. 6A, when a signal 6A related to an action of a user received from the wearable device 1 matches with an envelope area between upper and lower envelopes 5A and 5B related to a predetermined action, the control unit 20 may determine that an action corresponding to the envelopes 5A and 5B is performed by the user.

In addition, the control unit 20 may determine the degree of accuracy or similarity of the corresponding action and display the determined degree of accuracy or similarity through the display unit 23 such that the degree of accuracy or similarity is viewed by a user. That is, when a signal 6B related to an action of a user matches with the envelope area between the upper envelope 5A and the lower envelope 5B related to the predetermined action (for example, at a ratio greater than or equal to 70%) the control unit 20 may determine that an action corresponding to the envelope area is performed and may determine the degree of accuracy or similarity using the degree to which the corresponding signal 6B matches with the envelope area between the upper envelope 5A and the lower envelope 5B.

In the case of FIG. 6B, for example, when the signal 6B related to the action of the user matches with the envelope area between the upper envelope 5A and the lower envelope 5B at a ratio of 80%, the control unit 20 may determine that the user performs an action corresponding to the envelopes 5A and 5B with a degree of accuracy of 80% and display a result of the determination to be checked by the user.

That is, the control unit 20 may accurately recognize an action of a user as in the above and determine the degree of accuracy or similarity of the action of the user. Accordingly, the control unit 20 may determine calories consumed by the action of the user and may display the consumed calories to the user through the display unit 23.

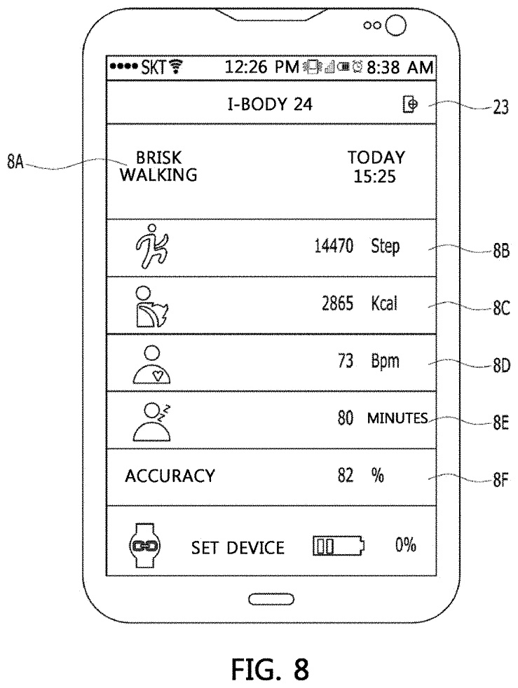

FIG. 8 is a diagram for describing an example of implementation according to the present invention. As shown in FIG. 8, the control unit 20 may recognize an action of the user 8A according to a degree of match with the envelope area, display the amount of exercise of the user 8B, the consumed calories 8C, the maximum heart rate 8D, the exercise time 8E, and the degree of accuracy or similarity 8F through the display unit 23 of the mobile terminal 2.

In the embodiment of the present invention, only a single envelope area is described as being used for determining an action of a user, but the present invention is not limited thereto. For example, in the case of climbing a mountain, a signal measured by the altitude sensor 13 and a signal measured by the acceleration sensor 12 may constitute a signal related to an action of a user, and in this case, the plurality of signals may be compared with the envelopes stored in the storage unit 24 to determine the action of the user.

Meanwhile, in the case of a complicated action, such as football, the control unit 20 may determine the action of the user through a combination of various actions. For example, the control unit 20 may determine each action of walking, running, jumping, and stabilization and identify an action from a combination of the respective actions. Alternatively, in the case of golf, which is composed of a combination of a plurality of actions, the storage unit 24 may store an envelope and an envelope area that are composed of the combination of the plurality of actions.

FIG. 7 is a diagram for describing an acceleration pattern corresponding to golf according to an embodiment of the present invention.

As shown in the drawing, the pattern of the acceleration signal is clearly distinguished for each step of a swing, and the control unit 20 may receive a signal related to an action of a user received from the wearable device 1 as shown in FIG. 7. Then, the control unit 20 may compare the signal with an envelope area related to a golf swing stored in the storage unit 24 and determine whether the action corresponds to a golf swing and may determine the degree of accuracy or similarity of the action.

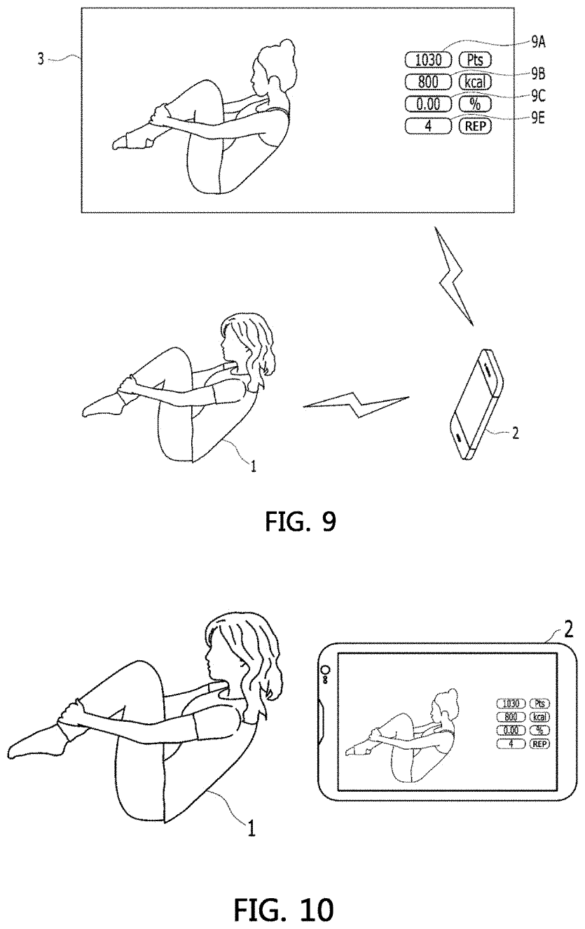

FIG. 9 is a structural view illustrating the fitness monitoring system according to the embodiment of the present invention.

As shown in the drawing FIG. 9, the fitness monitoring system according to the present invention may include the display apparatus 3, the wearable device 1 worn by a user, and the mobile terminal 2.

The user may perform an operation of a trainer as displayed on the display apparatus 3 while wearing the wearable device 1. In this case, the display apparatus 3 may display an image that is received from the mobile terminal 2 in real time. The display apparatus 3 may be a television, but the present invention is not limited thereto. For example, the display apparatus 3 may be a display unit of a personal computer or a display unit of a notebook computer. Alternatively, the display apparatus 3 may be a pad-type computer.

To this end, the communication unit 21 of the mobile terminal 2 may provide the image by performing communication with the display apparatus 3 in a predetermined communication scheme. The communication unit 21 may perform communication with the display apparatus 3 by a short-range communication scheme, or a communication scheme for a home network. In this case, the communication unit 21 may further include a module for home network communication in addition to a module for short-range communication.

In this case, an image being displayed on the display apparatus 3 may also be displayed on the mobile terminal 2, or the image may only be displayed on the display apparatus 3.

However, the present invention is not limited thereto, and the system according to the present invention may only include the wearable device 1 and the mobile terminal 2.

FIG. 10 is a structural view illustrating a fitness monitoring system according to another embodiment of the present invention.

As shown in the drawing, a user may view an operation of a trainer through the display unit 23 of the mobile terminal 2.

Since the system shown in FIG. 10 is identical to that shown in FIG. 9 except that an image received by the mobile terminal 2 is displayed through the display unit 23 or directly displayed through the mobile terminal 2, the following description will be made with reference to the system shown in FIG. 9 for the sake of convenience in description.

A user wearing the wearable device 1 may select one of various fitness operations stored in the storage unit 24 of the mobile terminal 2. For example, the types of actions stored in the storage unit 24 may be stored in a tree structure as follows.

TABLE 1

| Main classification | Medium classification | Minor classification |
|---|---|---|
| Basic exercises | Warm-up | Neck stretching |
| | | Wrist stretching |
| | | Waist bending with arms |
| | | backward |
| | | Side stretching |
| | | Shoulder stretching |
| | | Windmill exercise |
| | | Waist bending |
| | Whole body exercise | Arms and legs shaking |
| | | Push-up |
| | | High jump in place |
| | | Squat |
| | | Jump in place |
| | | Crunch |
| | | Side kick |
| | | Side lunge |
| | | Jumping jack |
| | | Back and chest exercise |
| | Mat exercise | Rolling like a ball |
| | | Open leg rocker |
| | | Double leg pull |
| | | Squat |
| | | Swimming |
| | Active exercise | Side step jacks |
| | | Criss cross squat |
| | | Curtsy lunge |
| | | Alt toe tap squat |
| | | Squat hops |
| Fitness program | Pilates | Wide squat pile |
| | | Roll up |
| | | Double leg circle |
| | | C-curved crunch |
| | | Heel touch |
| | | Criss cross |
| | | Oblique side band |
| | | O balance |
| | | Plank push up |
| | Muscle | Jumping jack |
| | | Arm walking |
| | | High knee freeze |
| | | Hip bridge |
| | | Squat hop |
| | | Push-up |
| | | Heel touch |
| | | Reverse lunge |
| | | Sky reach |
| | | Sumo walk |

However, this should be considered illustrative, and the present invention is not limited to storing the above-described operations and may store more various actions. In addition, such operations are not separately stored so that when an exercise corresponding to the medium classification is selected, an operation in the minor classification may be continuously displayed.

In addition, the storage unit 24 may store an upper envelope, a lower envelope, and an envelope area between the upper and lower envelopes to correspond to such an operation. That is, for the embodiment of the present invention, a server (not shown) for providing the fitness monitoring system according to the present invention may measure a signal related to an action of a trainer multiple times, determine an upper envelope and a lower envelope for the signals as shown in FIG. 5, and store the upper and lower envelopes and the envelope area to correspond to each exercise.

When a user selects an operation, an image in which the trainer performs the operation may be displayed on the display apparatus 3 as shown in FIG. 9. The image may be one of the images stored in the storage unit 24 of the mobile terminal 2, which is transmitted from the control unit 20 to the display apparatus 3 through the communication unit 21 as described above. Alternatively, the image may be displayed on the display unit 23 of the mobile terminal 2 as shown in FIG. 10.

As the user checks the image displayed on the display unit 23 and follows the operation, a signal related to an action of the user is transmitted from the wearable device 1 worn by the user to the mobile terminal 2.

The control unit 20 of the mobile terminal 2 may compare the envelope area of the operation with the signal related to the action transmitted from the wearable device of the user and determine the degree of accuracy or similarity of the operation. A method of determining the degree of accuracy or similarity may be achieved by checking a ratio corresponding to a degree of match to which the signal related to the action of the user matches with (coincides with) the envelope area between the upper and lower envelopes 5A and 5B as shown in FIG. 6B.

In addition, the control unit 20 may assign points depending on the degree of accuracy or similarity of the operation and may determine calories consumed by the operation. In addition, the control unit 20 may display the number of times the operation is performed.

That is, the control unit 20 may display the points 9A, the calorie consumption 9B, the degree of accuracy 9C, and the operation execution repetition number 9D in a portion of the image of the trainer such that the user is provided with convenience in checking his or her operation.

The above has been described by way of an example in which the wearable device 1 transmits a signal related to an action performed by a user to the mobile terminal 2, and the mobile terminal 2 receives the signal related to the action of the user to identify the action depending on the degree to which the signal corresponds to the envelope area and determine the degree of accuracy or similarity of the action. However, the above should be regarded as illustrative, and the wearable device 1 may collect a signal related to an action performed by the user, identify the action depending on the degree to which the signal corresponds to the previously stored envelope area, determine the degree of accuracy or similarity of the action, and transmit the determined degree of accuracy or similarity of the action to the mobile terminal 2.

In this case, the storage unit for storing envelope areas for a plurality of actions and the control unit for comparing a signal related to an action of a user with the envelope area, determining an action corresponding to the envelope area, and determining the degree of accuracy or similarity of the action of the user may be provided in the wearable device 1.

In this case, the action of the user and the degree of accuracy or similarity of the action may be transmitted to the mobile terminal 2, and the type of the action and the degree of accuracy or similarity of the action may be displayed on the display unit 23 of the mobile terminal 2.

According to the present invention, a predetermined operation can be identified from a signal related to an action of a user depending on whether the signal falls within a signal range corresponding to the predetermined operation, and the degree of accuracy of the operation can be determined by checking a ratio corresponding to deviation of the signal from the signal range corresponding to the predetermined operation.

Thus, according to the present invention, an operation corresponding to an action of a user can be more accurately identified, and the user is guided to more accurately perform the operation by providing the degree of accuracy so that the exercise efficiency is enhanced.

Although the embodiments of the present invention have been described with reference to the accompanying drawings, this is for illustrative purposes, and a person of ordinary skill in the art should appreciate that various modifications, equivalents, and other embodiments are possible without departing from the scope and sprit of the present invention. Therefore, the scope of the present invention is defined by the appended claims of the present invention.

1. A fitness monitoring system comprising:
a wearable device worn on a predetermined portion of a body of a user to transmit a signal related to an action of the user; and
a mobile terminal configured to determine the action of the user on the basis of the signal related to the action of the user,
wherein the mobile terminal includes:
a storage unit configured to store an envelope area that is determined by connecting ends of a plurality of signals corresponding to a predetermined action; and
a control unit configured to determine the action of the user as the predetermined action when the signal related to the action of the user corresponds to the envelope area.

2. The fitness monitoring system of claim 1, wherein the signal related to the action of the user is an acceleration signal.

3. The fitness monitoring system of claim 1, wherein the control unit checks and determines whether a plurality of actions each correspond to the envelope area and determines the action of the user on the basis of a combination of the plurality of actions.

4. The fitness monitoring system of claim 1, wherein the control unit determines a degree of accuracy or similarity of the action of the user using a degree to which the signal related to the action of the user matches with the envelope area.

5. A fitness monitoring system comprising:
a wearable device worn on a predetermined portion of a body of a user to transmit a signal related to an action of the user; and
a mobile terminal configured to display an image in which a trainer performs an operation, determine the action of the user on the basis of the signal related to the action of the user, and determine a degree of accuracy or similarity of the action of the user,
wherein the mobile terminal includes:
a display unit;
a storage unit configured to store the image in which the trainer performs the operation and an envelope area of a signal corresponding to the operation of the trainer; and
a control unit configured to control the display unit to display the image of the trainer and determine a degree to which the signal related to the action of the user received from the wearable device matches with the envelope area.

6. The fitness monitoring system of claim 5, wherein the signal related to the action of the user is an acceleration signal.

7. (canceled)

8. (canceled)

9. A fitness monitoring system comprising:
a wearable device worn on a predetermined portion of a body of a user to collect a signal related to an action of the user, determine information related to the action of the user on the basis of the signal related to the action of the user, and transmit the information related to the action of the user; and
a mobile terminal configured to receive the information related to the action of the user,
wherein the wearable device includes:
a sensor unit configured to collect the signal related to the action of the user;
a storage unit configured to store an envelope area that is determined by connecting ends of a plurality of signals corresponding to a predetermined action; and
a control unit configured to determine the action of the user as the predetermined action when the signal related to the action of the user corresponds to the envelope area.

10. The fitness monitoring system of claim 9, wherein the control unit checks and determines whether a plurality of actions each correspond to the envelope area and determines the action of the user on the basis of a combination of the plurality of actions.

11. The fitness monitoring system of claim 9, wherein the control unit determines a degree of accuracy or similarity of the action of the user using a degree to which the signal related to the action of the user matches with the envelope area.
